(19) United States
(12) Patent Application Publication
(10) Pub. No.: US 2016/0101266 A1
(43) Pub. Date: Apr. 14, 2016

(54) Medical Instrument Clamp

(71) Applicant: Onanon, Inc., Milpitas, CA (US)

(72) Inventors: Dennis Joel Johnson, Milpitas, CA (US); Maziyar Keshtgar, Milpitas, CA (US); Alex Pruitt, Milpitas, CA (US); Katie Hoose, Milpitas, CA (US)

(73) Assignee: Onanon, Inc.

(21) Appl. No.: 14/510,805

(22) Filed: Oct. 9, 2014

(51) Int. Cl. A61M 25/09; A61B 19/00

(52) U.S. Cl. CPC A61M 25/09; A61B 19/26; A61B 2019/267; A61M 2025/09125

(57) ABSTRACT

A medical instrument clamp for removably securing an elongated medical instrument within a housing to be conductively connected to a conduit. The present invention generally includes a housing having a first opening at its first end and a second opening at its second end. The first opening leads to a first channel adapted to receive an elongated medical instrument such as a catheter guide wire. The second opening leads to a second channel adapted to receive a conduit such as a connection conduit for a medical device. A contact internal to the housing is positioned between the first and second channels to conductively connect the medical instrument with the conduit. A pair of arms and clamp are included which may be transitioned to open or close in response to manipulation of a knob. By closing the arms and clamp, the medical instrument may be removably secured within the first channel.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to a clamping device and more specifically it relates to a medical instrument clamp for removably securing an elongated medical instrument such as a catheter guide wire within a housing such that the medical instrument is conductively connected to a conduit.

2. Description of the Related Art

Any discussion of the related art throughout the specification should in no way be considered as an admission that such related art is widely known or forms part of common general knowledge in the field.

Elongated medical instruments such as catheter guide wires and various probes are commonly used in the medical field. Such instruments are almost universally used in most surgical procedures and often for routine care. These medical instruments are often difficult to manipulate and generally come with their own housings. Due to the nature of many of these medical instruments, they must be disposed of after usage. The disposal of an integral housing can be wasteful, particularly in a cost-sensitive field such as health care.

It is also often necessary to connect the medical instrument to a medical device such as a monitor or to a source of power. This can increase the cost of the integral housing often included with such instruments and thus greatly increase the costs associated with the disposal of such medical instruments. It would be far preferable to include a reusable base housing which may be re-used to connect disposable or cleanable medical instruments to a power supply or medical device.

Because of the inherent problems with the related art, there is a need for a new and improved medical instrument clamp for removably securing an elongated medical instrument such as a catheter guide wire within a housing such that the medical instrument is conductively connected to a conduit.

### BRIEF SUMMARY OF THE INVENTION

The invention generally relates to a clamping device which includes a housing having a first opening at its first end and a second opening at its second end. The first opening leads to a first channel adapted to receive an elongated medical instrument such as a catheter guide wire. The second opening leads to a second channel adapted to receive a conduit such as a connection conduit for a medical device. A contact internal to the housing is positioned between the first and second channels to conductively connect the medical instrument with the conduit. A pair of arms and clamp are included which may be transitioned to open or close in response to manipulation of a knob. By closing the arms and clamp, the medical instrument may be removably secured within the first channel.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is an exploded upper perspective view of the present invention.

FIG. 3 is an exploded top view of the present invention.

FIG. 4a is a side sectional view of the present invention.

FIG. 4b is an exploded side sectional view of the present invention.

FIG. 5 is a side sectional view of the present invention with medical instrument and conduit installed.

FIG. 6 is a frontal upper perspective view of the present invention with medical instrument and conduit installed.

FIG. 7 is a frontal view of the knob of the present invention.

FIG. 8a is a frontal view of the present invention in an opened state.

FIG. 8b is a frontal view of the present invention in a closed state.

FIG. 9 is a top view of the present invention illustrating alignment of a medical instrument prior to insertion.

FIG. 10a is a frontal sectional view of the present invention in an opened state.

FIG. 10b is a frontal sectional view of the present invention in a closed state.

FIG. 11 is a side sectional view of a contact for use with the present invention.

FIG. 12 is a side sectional view of a contact with a medical instrument installed in its receiver portion and its lead portion connected to a conduit.

FIG. 13a is an upper perspective view of a first alternate embodiment of the knob of the present invention in a closed position.

FIG. 13b is an upper perspective view of a first alternate embodiment of the knob of the present invention in an opened position.

FIG. 14a is an upper perspective view of a second alternate embodiment of the knob of the present invention in an opened position.

FIG. 14b is an upper perspective view of a second alternate embodiment of the knob of the present invention in a closed position.

FIG. 15a is a sectional view of the first alternate embodiment of the knob of the present invention shown in FIG. 13a.

FIG. 15b is a sectional view of the first alternate embodiment of the knob of the present invention shown in FIG. 13b.

FIG. 16a is a sectional view of the second alternate embodiment of the knob of the present invention shown in FIG. 14a.

FIG. 16b is a sectional view of the second alternate embodiment of the knob of the present invention shown in FIG. 14b.

### DETAILED DESCRIPTION OF THE INVENTION

### A. Overview.

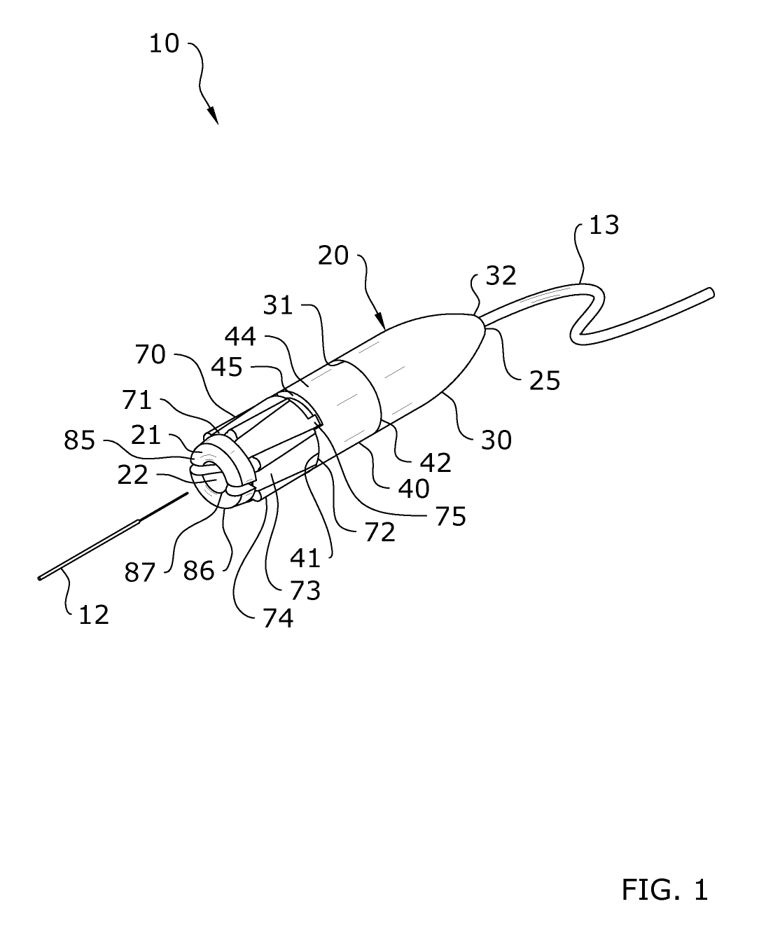

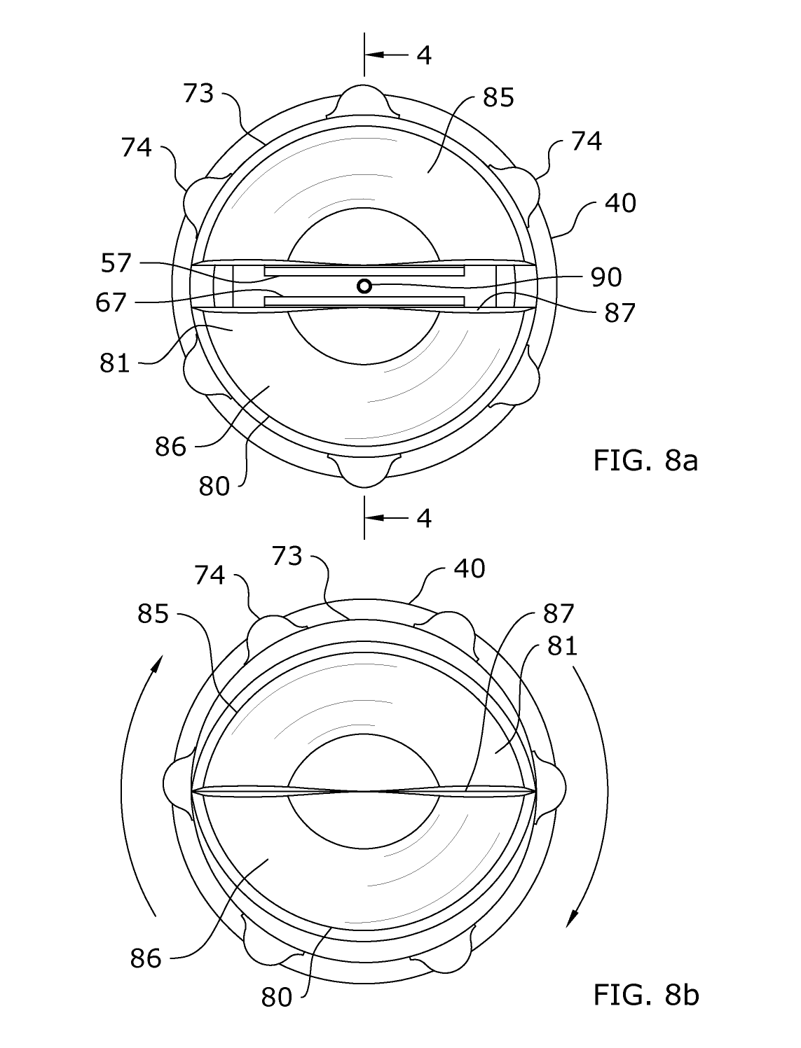

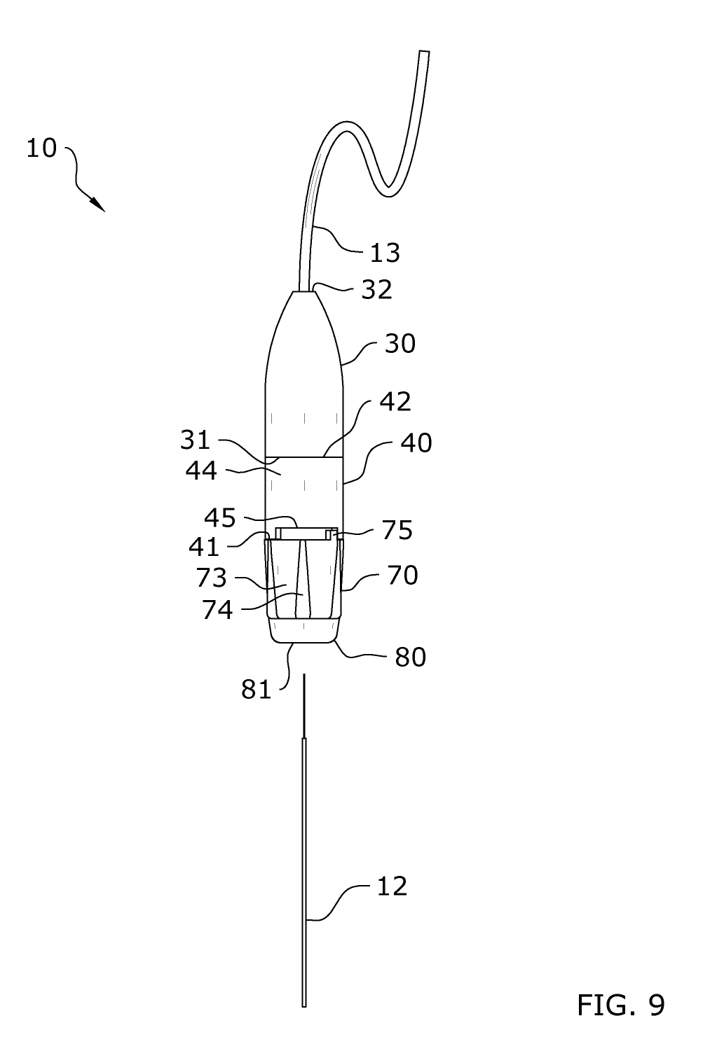

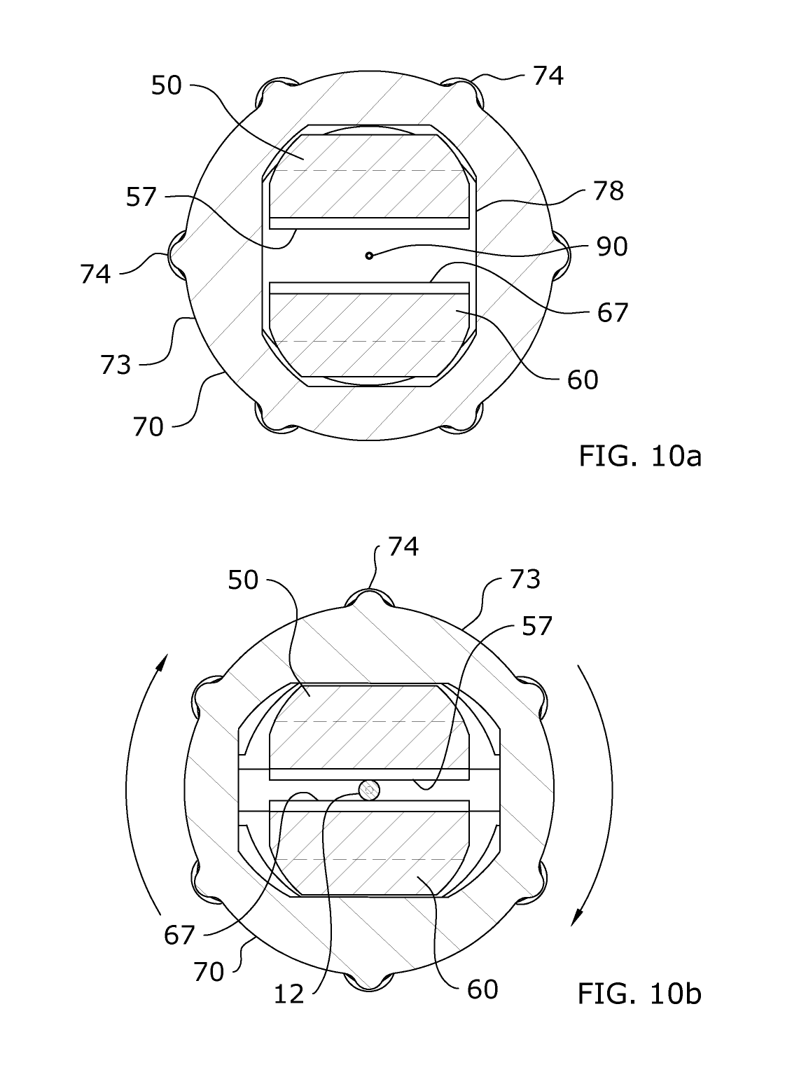

Turning now descriptively to the drawings, in which similar reference characters denote similar elements throughout the several views, FIGS. 1 through 16b illustrate a medical instrument clamp 10, which comprises a housing 20 having a first opening 22 at its first end 21 and a second opening 26 at its second end 25. The first opening 22 leads to a first channel 23 adapted to receive an elongated medical instrument 12 such as a catheter guide wire. The second opening 26 leads to a second channel 27 adapted to receive a conduit 13 such as a connection conduit for a medical device. A contact 90 internal to the housing 20 is positioned between the first and second channels 23, 27 to conductively connect the medical instrument 12 with the conduit 13. A pair of arms 50, 60 and clamp 80 are included which may be transitioned to open or close in response to manipulation of a knob 70. By closing the arms 50, 60 and clamp 80, the medical instrument 12 may be removably secured within the first channel 23.

### B. Housing.

As best shown in FIGS. 1-3, the present invention generally comprises a housing 20 having a first end 21 and a second end 25. It should be appreciated that the shape, size, and configuration of the housing 20 may vary in different embodiments and thus should not be construed as being limited by the exemplary embodiment shown in the figures.

The embodiment shown in the figures comprises a base portion 30, a linkage portion 40, a knob 70, and a clamp 80. While these components of the present invention are shown in the exemplary figures as being comprised of interconnected, discrete structures, it should be appreciated that one or more of any of these components may be integrally formed with one or more any remaining components. By way of example, the base portion 30 could be integrally formed with the linkage portion 40.

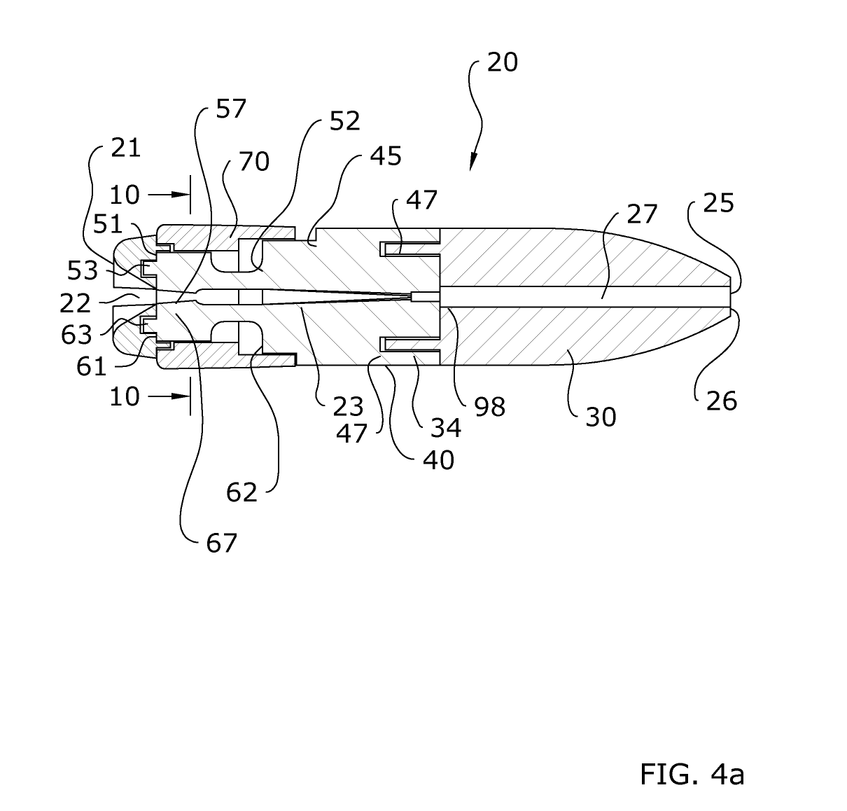

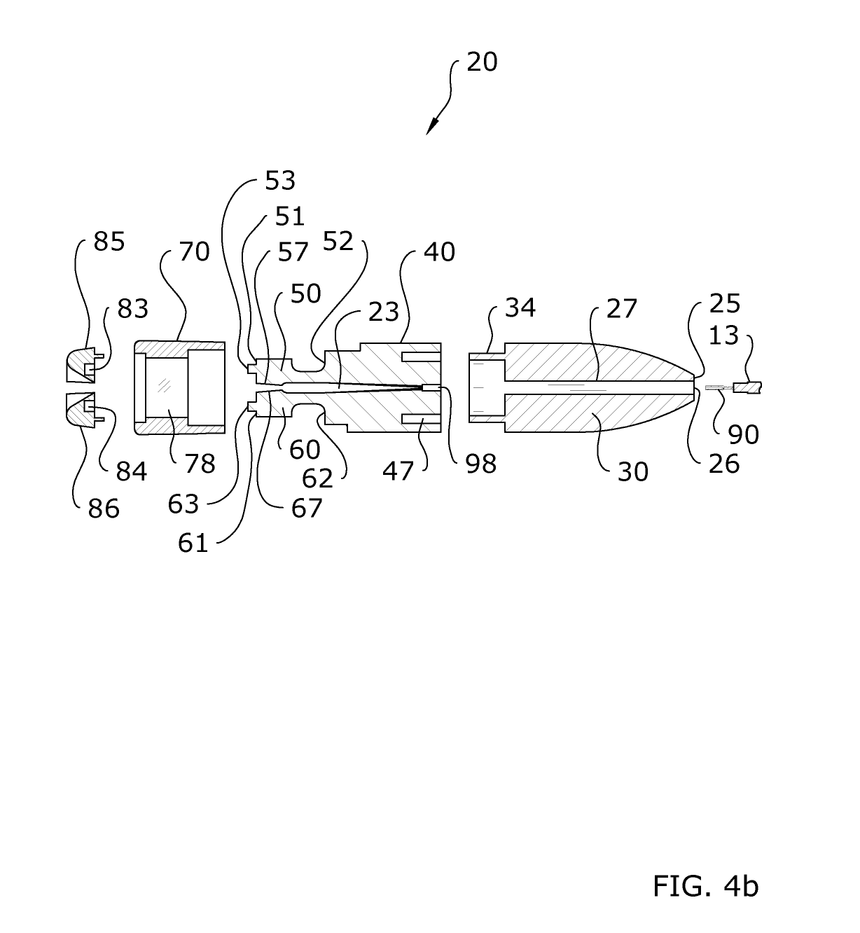

As best shown in FIG. 4a, the first end 21 of the housing 20 includes a first opening 22 leading to a first channel 23. The first channel 23 extends from the first opening 22 to the approximate mid-point of the housing 20. The second end 25 of the housing 20 includes a second opening 26 leading to a second channel 27. The second channel 27 extends from the second opening 26 to the approximate mid-point of the housing 20. The first and second channels 23, 27 are generally connected by a contact 90 and/or connector hub 95.

The first opening 22 is adapted to receive a medical instrument 12 which may be extended through the first channel 23 to be conductively connected to a contact 90 which is internal to the housing 20. Through use of a knob 70, the first channel 23 may be selectively closed around the medical instrument 12 to secure the medical instrument 12 within the first channel 23.

The second opening 26 is adapted to receive a conduit 13 which generally extends through the second channel 27 and is connected to a contact 90. In some embodiments, the conduit 13 may be connected to a connector hub 95 which is conductively connected to the contact 90. The conduit 13 may be fixedly or removably secured within the second channel 27.

i. Base Portion.

The housing 20 includes a base portion 30 best shown in FIGS. 1-3. The base portion 30 includes a first end 31 and a second end 32. The size, shape, and configuration of the base portion 30 may vary in different embodiments. The exemplary embodiment shown in the figures, illustrating a base portion 30 which tapers in width between its first and second ends 31,32 should not be construed as limiting on the scope of the present invention.

The first end 31 of the base portion 30 is connected to the second end 42 of the linkage portion 40. The second end 32 of the base portion 30 includes the second opening 26 of the housing 20. The second channel 27 of the housing 20 also extends through the base portion 30 between its first and second ends 31, 32 as best shown in FIG. 4.

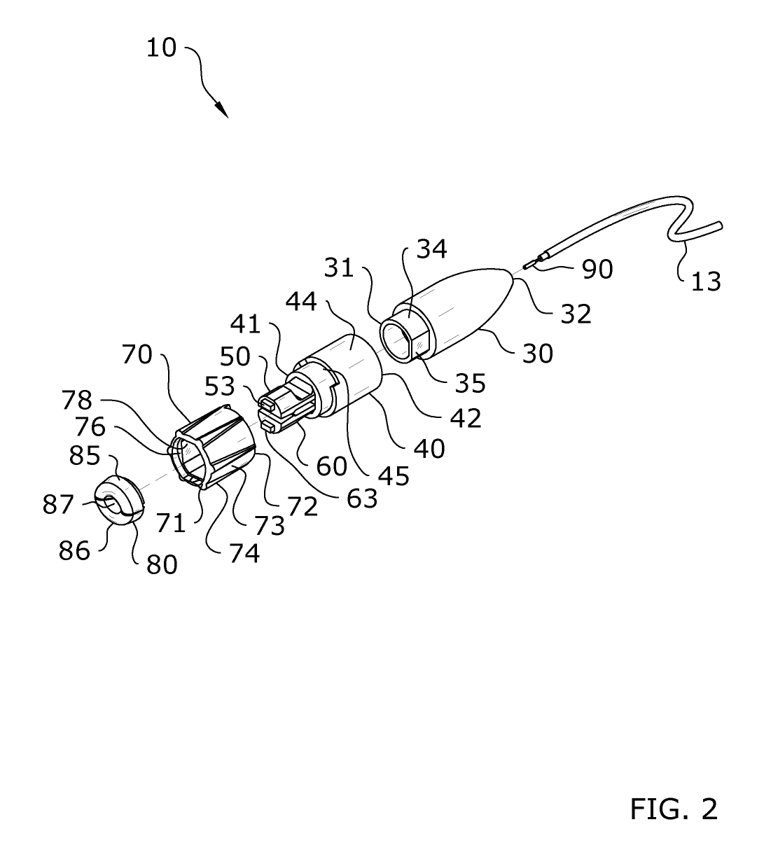

It is appreciated that, in some embodiments, the base portion 30 and linkage portion 40 may be integrally formed. Generally, the base portion 30 will not be rotatable but will be fixed in place. In an embodiment with discrete, interconnected base and linkage portions 30, 40 as best shown in FIG. 2, the base portion 31 may include a connector 34 at its first end 31.

The connector 34 may be comprised of any structure which may interconnect the base portion 30 with the linkage portion 40 of the housing 20. In the embodiment shown in the figures, the connector 34, comprised of a rim extending from the first end 31 of the base portion 30, will frictionally fit within a receiver 47 at the second end 42 of the linkage portion 40. It is preferable that the base portion 30 be removable from the linkage portion 40, but there will preferably be a firm, frictional engagement to prevent the two portions 30, 40 from becoming disconnected inadvertently. The use of a flattened portion 35 on the connector 34 may prevent rotation of the base portion 30 with respect to the linkage portion 40.

ii. Linkage Portion.

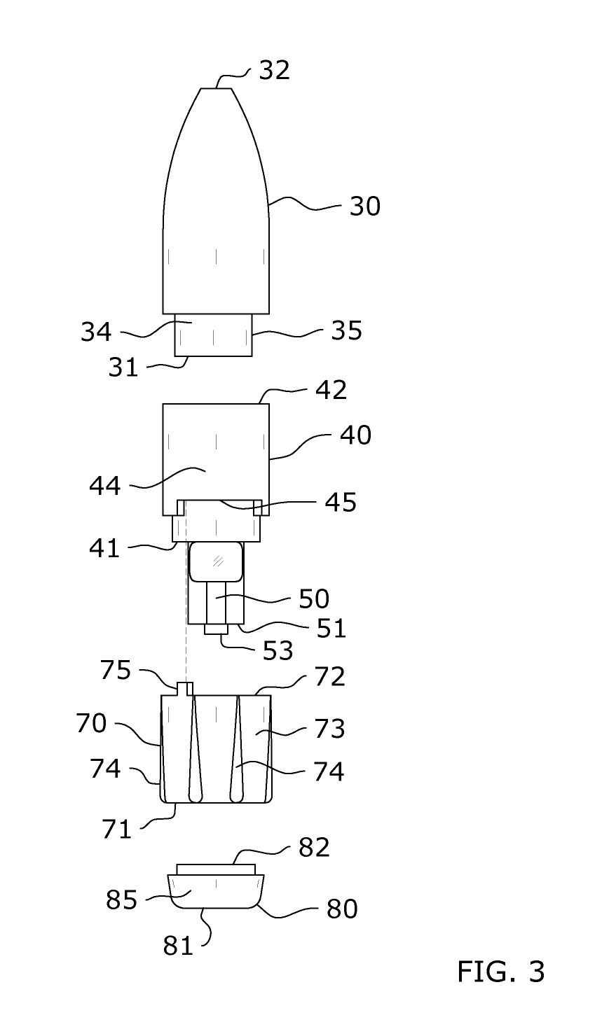

The linkage portion 40 of the housing 20 is best shown in FIGS. 2-3. As shown, the linkage portion 40 includes a first end 41 and a second end 42. The linkage portion 40 is connected at its second end 42 to the base portion 30. The knob 70 and clamp 80 of the present invention are positioned over the second end 42 of the linkage portion 40.

As shown in FIG. 2, the linkage portion 40 comprises a gripping portion 44 at its second end 42 and a pair of arms 50, 60 extending from its first end 41. The first channel 23 of the housing 20 extends through the linkage portion 40. The first and second arms 50, 60 aid in securing the medical instrument 12 within the housing 20 by closing the first channel 23 around the medical instrument 12 when actuated as discussed herein.

The gripping portion 44 is comprised of a circular cross-section which may be gripped by the user when the present invention is in use. The gripping portion 44 includes a stopper slot 45 formed therein which is adapted to inhibit rotation of the knob 70 as will be described herein. The stopper slot 45 generally extends approximately sixty degrees around the outer circumference of the gripping portion 44 of the linkage portion 40 of the housing 20.

The gripping portion 44 also includes a receiver 47 previously discussed which frictionally receives the connector 34 of the base portion 30 to connect the base and linkage portions 30, 40 together in embodiments in which they are not integrally formed. The receiver 47 will thus be generally comprised of an opening or slot at the second end 42 of the linkage portion 40 which has an inner circumference matching the outer circumference of the connector 34.

iii. Arms.

As best shown in FIGS. 4b, a pair of arms 50, 60 extend from the first end 41 of the linkage portion 40. The first channel 23 of the housing 20 is defined between the first arm 50, and the second arm 60 as shown in FIG. 4a. When the arms 50, 60 are pressed together via actuation of the clamp 80, the first channel 23 will close onto the medical instrument 12 to secure the medical instrument 12 within the housing 20.

The first arm 50 is comprised of an elongated arm-like structure having a first end 51 and a second end 52. The second end 52 of the first arm 50 extends from the first end 41 of the linkage portion 40, and may be integrally formed therewith. The second end 52 of the first arm 50 includes a first anchor 53 which is adapted to engage with a corresponding first receiver slot 83 on the clamp 80.

The second arm 60 is comprised of an elongated arm-like structure having a first end 61 and a second end 62 which runs parallel with respect to the first arm 50. The second end 62 of the second arm 60 extends from the first end 41 of the linkage portion 40, and may be integrally formed therewith. The second end 62 of the second arm 60 includes a second anchor 63 which is adapted to engage with a corresponding second receiver slot 84 on the clamp 80.

The anchors 53, 63 are best shown in FIGS. 4a and 4b. The anchors 53, 63 act to rotationally lock the clamp 80 in place so that rotation or other manipulation of the knob 70 may open and close the clamp 80. Thus, the anchors 53, 63 may be comprised of any structure capable of securing the clamp 80 to the arms 50, 60 while preventing rotation of the clamp 80 with respect to the linkage portion 40. In the exemplary embodiment shown in the figures, the anchors 53, 63 are illustrated as being comprised of rectangular-shaped members extending outwardly from the respective arms 50, 60 which engage in a male-female manner with corresponding receiver slots 83, 84 on the clamp 80.

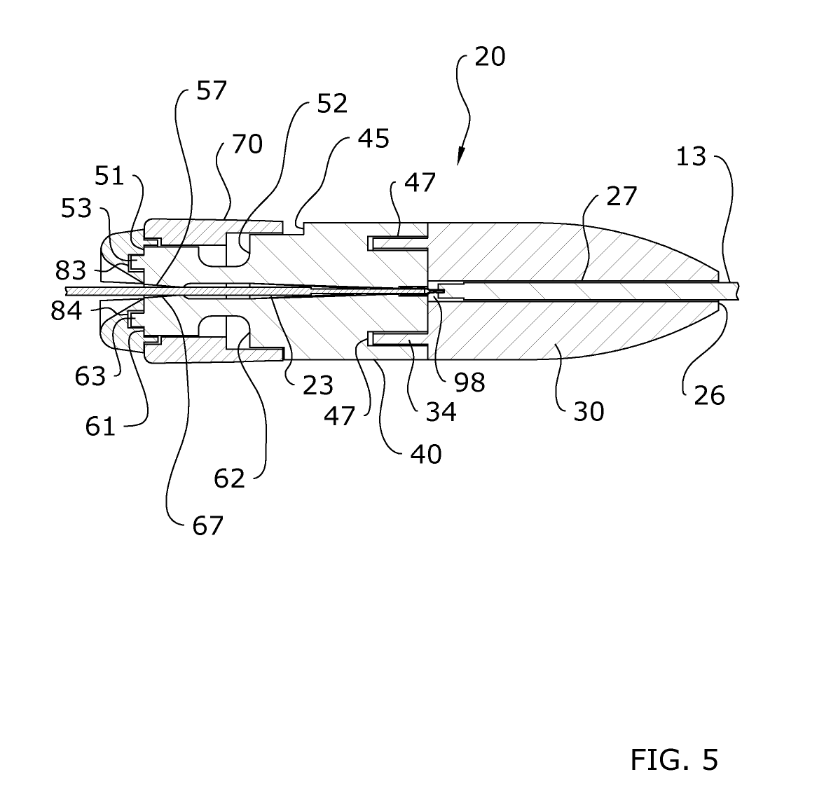

The first arm 50 also includes a first inner edge 57 which includes an extension into the first channel 23 adapted to press against the medical instrument 12 when the arms 50, 60 are closed together. The second arm 60 similarly includes a second inner edge 67 which includes an extension into the first channel 23 adapted to press against the medical instrument 12 when the arms 50, 60 are closed together. As best shown in FIG. 5, the first inner edge 57 will press downwardly against an upper end of the medical instrument 12 while the second inner edge 67 will press upwardly against a lower end of the medical instrument 12 to sandwich the medical instrument 12 between the first and second arms 50, 60.

iv. Knob.

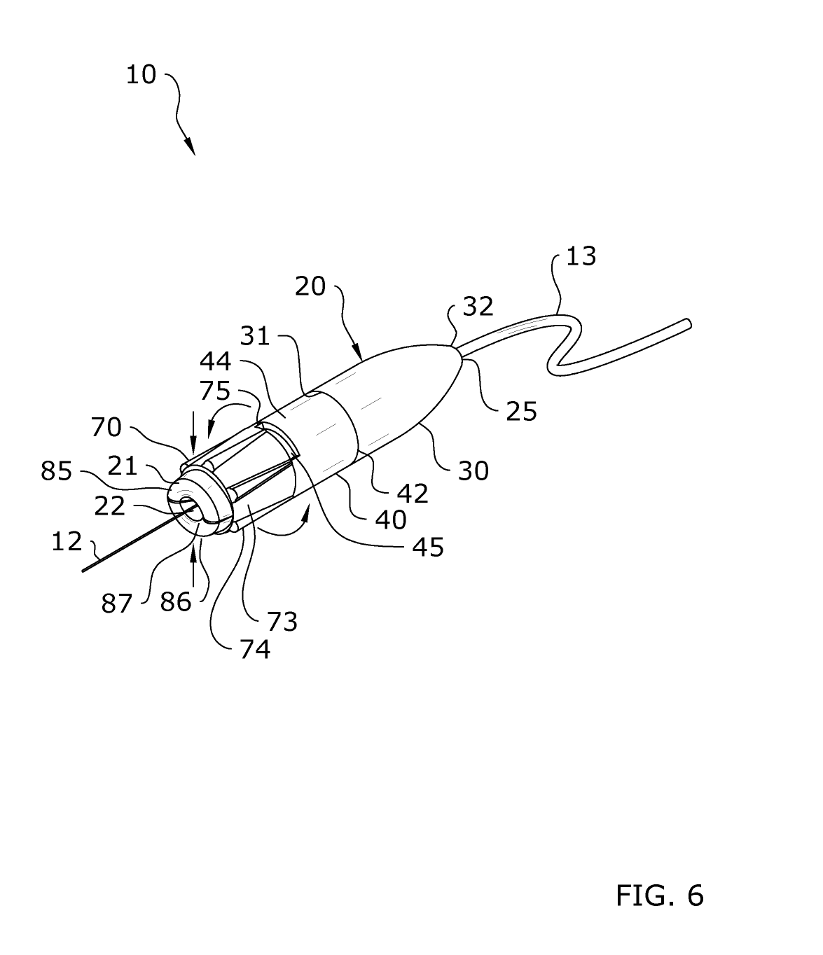

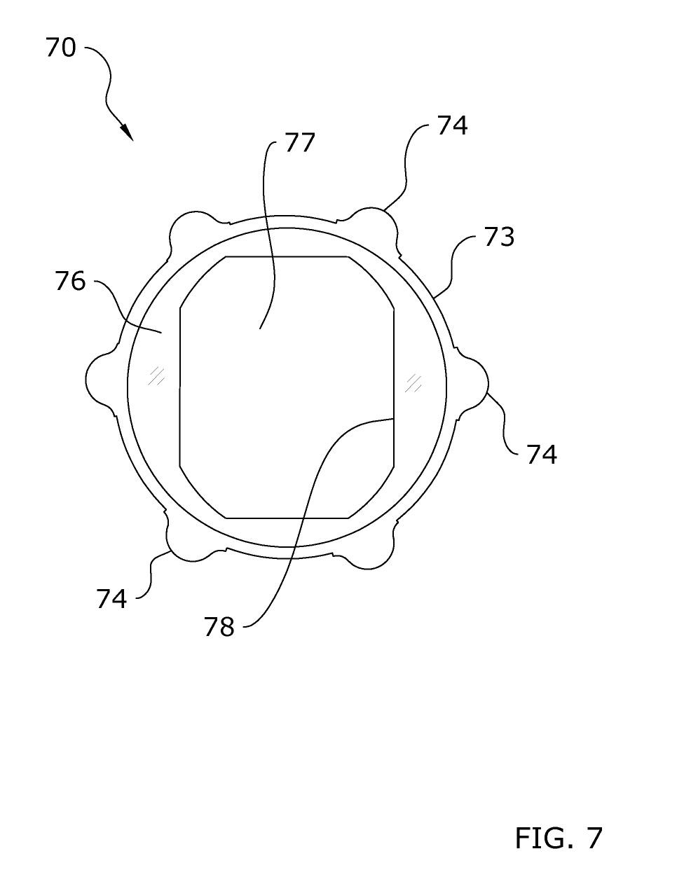

As shown throughout the figures, the housing 20 includes a knob 70 which is moved in a first direction to open the clamp 80 and arms 50, 60 and in a second direction to close the clamp 80 and arms 50, 60. A main embodiment of the knob 70 is best shown in FIGS. 6-7, and generally comprises a cylindrical member having a first end 71 and a second end 72. The knob 70 fits over the arms 50, 60 and linkage portion 40 in a rotational manner.

As best shown in FIG. 5, the knob 70 is generally comprised of a cylindrical member adapted to fit over and around the linkage portion 40 and arms 50, 60. The knob 70 is generally sandwiched between the clamp 80 and linkage portion 40 when the housing 20 is assembled, with the clamp 80 being positioned at the first end 71 of the knob 70.

The outer circumference 73 of the knob 70 may include one or more raised splines 74 extending between its first end 71 and second end 72 as shown in the figures. These raised splines 74 aid in gripping and rotating the knob 70 when the invention is in use. The splines 74 may be comprised of various configurations and may be omitted in some embodiments of the present invention.

The knob 70 also includes a locking tab 75 which extends from the outer circumference 73 of the knob 70 at its second end 72. The locking tab 75 is comprised of a tab or other extension which extends toward the linkage portion 40 from the second end 72 of the knob 70. The locking tab 75 is adapted to slide within the stopper slot 45 of the linkage portion 40. The locking tab 75 will be stopped by the stopper slot 45 when the knob 70 is rotated a certain distance in either direction. This will prevent full rotation of the knob 70 and aid in transitioning the clamp 80 between its open and closed positions.

The first end 71 of the knob 70 includes an inner lip 76 which extends inwardly from the inner circumference of the knob 70 at its first end 71 as best shown in FIG. 4b. The inner lip 76 will define a lip opening 77 in which the clamp 80 is positioned. The lip opening 77 is a different shape and smaller size than the hollow interior running through the cylindrical knob 70.

The inner surface 78 of the inner lip 76 will define the shape of the lip opening 77. The lip opening 77 should be shaped to allow the circular clamp 80 to be opened when the knob 70 is in a first position and closed when the knob 70 is in a second position. Thus, the lip opening 77 will preferably be comprised of an ovular shape or octagonal shape which will allow the circular clamp 80 to remain opened when the lip opening 77 is oriented in a first position and to close when the lip opening 77 is oriented in a second position, such as shown in FIGS. 7. Any other shape may be utilized so long as the clamp 80 is opened or closed in response to rotation of the knob 70.

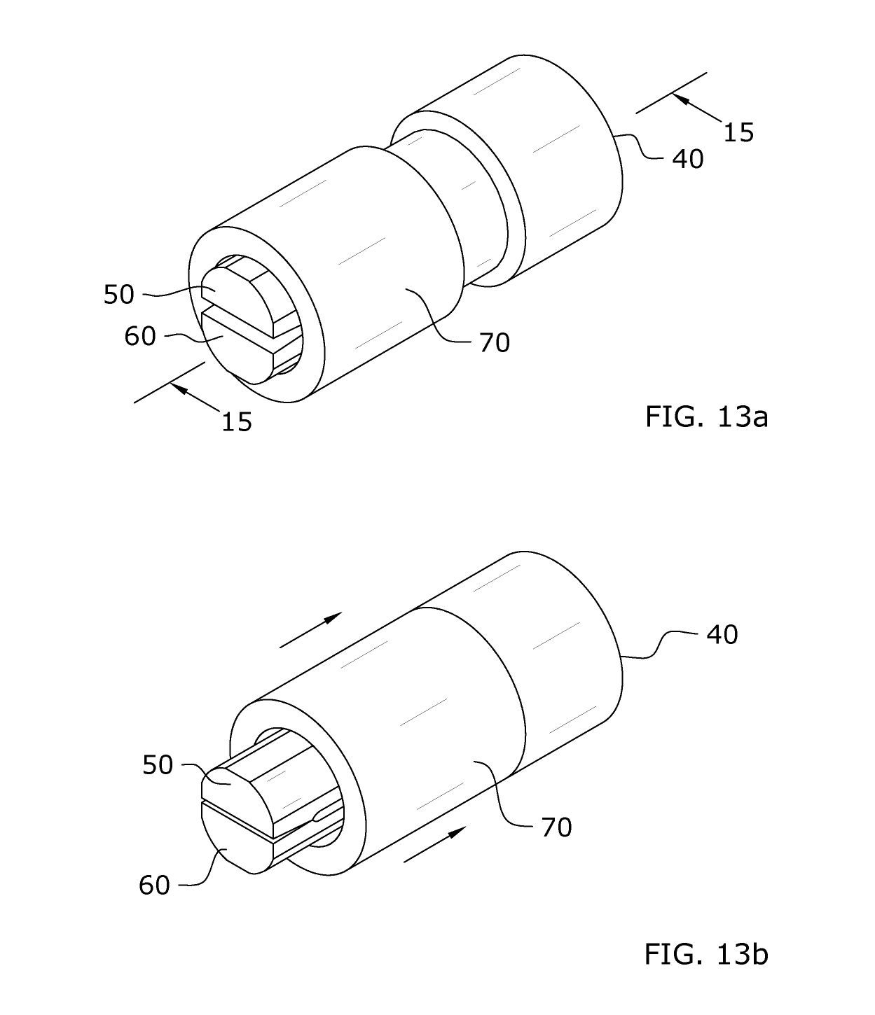

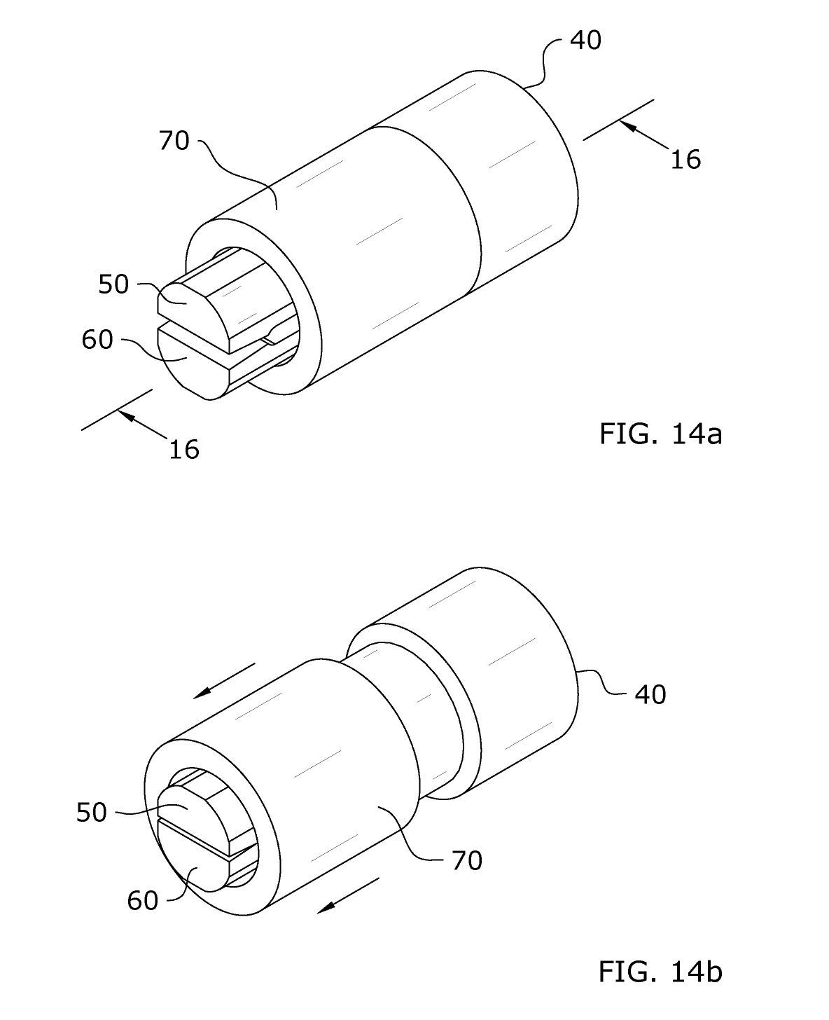

FIGS. 13a, 13b, 14a, and 14b illustrate alternate embodiments of the present invention in which the knob 70 is comprised of a sliding configuration instead of the rotating configuration shown in FIGS. 1-12. In such an embodiment, the knob 70 may be moved (such as by sliding) in a first direction to open the clamp 80 and in a second direction to close the clamp 80.

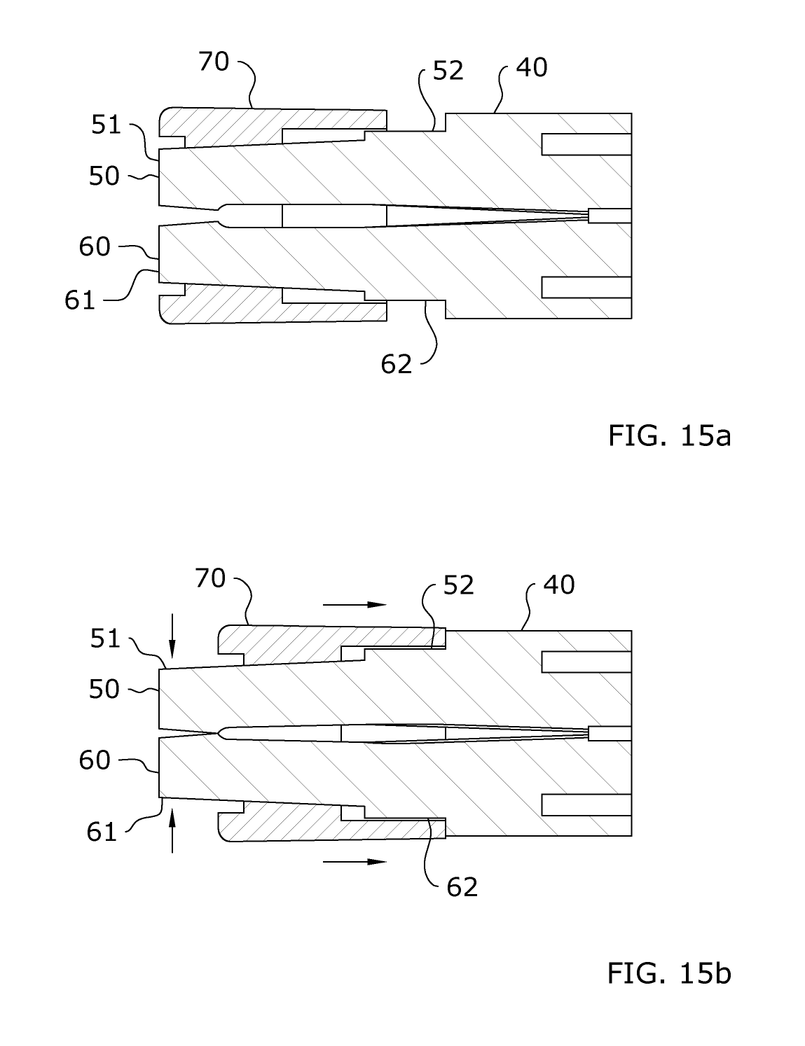

In FIGS. 13a and 13b, the knob 70 is pushed forward to open the clamp 80 and backward to close the clamp 80. FIGS. 15a and 15b illustrate a cross-sectional view in which it can be seen that the first arm 50 has an outer surface which tapers inwardly between its second end 52 and its first end 51 to create a ramp structure. The second arm 60 similarly has an outer surface which tapers inwardly between its second end 62 and its first end 61 to create a ramp structure. Thus, pulling the knob 70 backwards as shown in FIGS. 15a and 15b will cause the inner lip 76 of the knob 70 to force the arms 50, 60 against each other.

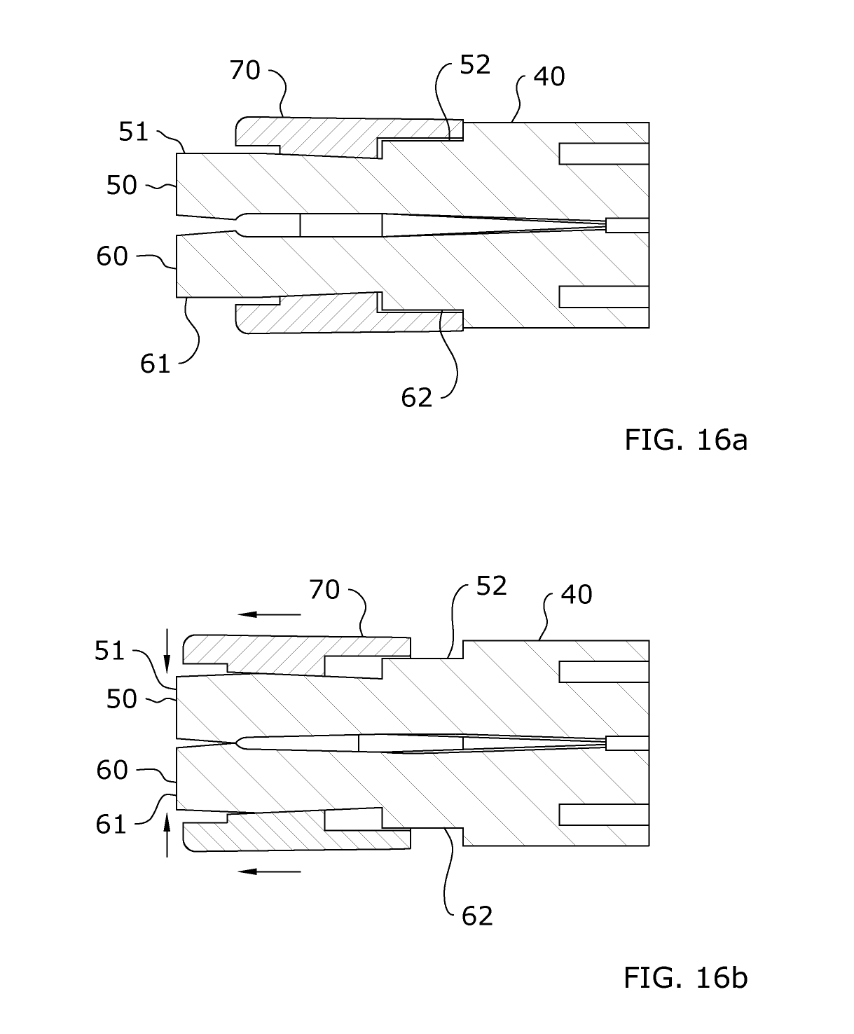

In FIGS. 14a and 14b, the knob 70 is pushed forward to close the clamp 80 and backward to open the clamp 80. FIGS. 16a and 16b illustrate a cross-sectional view in which it can be seen that the first arm 50 has an outer surface which tapers inwardly between tis first end 51 and its second end 52 to create a ramp structure. The second arm 60 similarly has an outer surface which tapers inwardly between its first end 61 and its second end 62 to create a ramp structure. Thus, pushing the knob 70 forwards as shown in FIGS. 15a and 15b will cause the inner lip 76 of the knob 70 to force the arms 50, 60 against each other.

v. Clamp.

As shown throughout the figures, the housing 20 includes a clamp 80 at its first end 21 which acts to retain the medical instrument 12 within the housing 20 when the knob 70 is in the closed position. The clamp 80 generally has a circular cross-section and has a first end 81 which includes a receiver opening 87 which corresponds with the first opening 22 of the housing 20. This receiver opening 87 is adapted to receive the medical instrument 12 and may be selectively opened or closed through actuation of a pair of jaws 85, 86 positioned at the first end 81 of the clamp.

The jaws 85, 86 act to close down upon the medical instrument 12 to lock the medical instrument 12 in the housing 20. The first jaw 85 extends downwardly upon the medical instrument 12 and the second jaw 86 extends upwardly upon the medical instrument 12 so that it is retained therebetween. In combination with the arms 50, 60, the jaws 85, 86 will ensure a tight and secure fit of the medical instrument 12 within the housing 20. The structure of the jaws 85, 86 may vary in different embodiments, so long as they are operable to close down around the medical instrument 12 in a first position and release the medical instrument 12 in a second position.

As best shown in FIGS. 4a and 4b, the second end 82 of the clamp 80 includes a pair of receiver slots 83, 84. A first receiver slot 83 positioned behind the first jaw 85 is adapted to receive and engage with the first anchor 53 of the first arm 50. A second receiver slot 84 positioned behind the second jaw 86 is adapted to receive and engage with the second anchor 63 of the second arm 60. The connection between the anchors 53, 63 and receiver slots 82, 83 will prevent rotation of the clamp 80 and ensure that the jaws 85, 86 open and close with the arms 50, 60 of the linkage portion 40.

### C. Contact.

The housing 20 includes an internal contact 90 which will conductively connect the medical instrument 12 positioned within the first channel 23 with a conduit 13 positioned within the second channel 27 as shown in FIG. 5. Any type of contact 90 capable of conductively connecting a space between two channels 23, 27 may be utilized. For example and without limitation, the contact 90 may be comprised of a bifurcated configuration, pogo configuration, trifurcated configuration, or a leaf spring configuration.

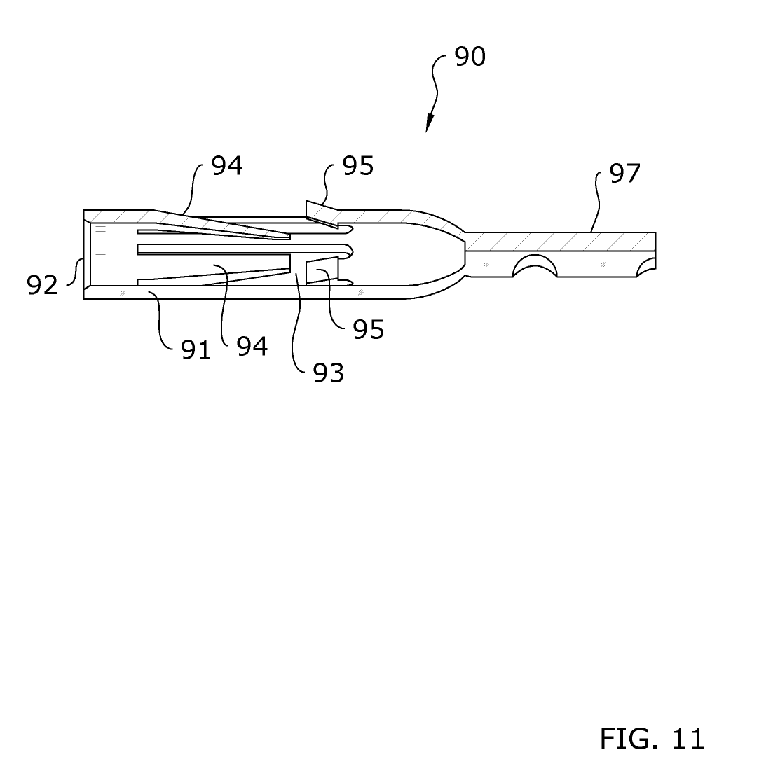

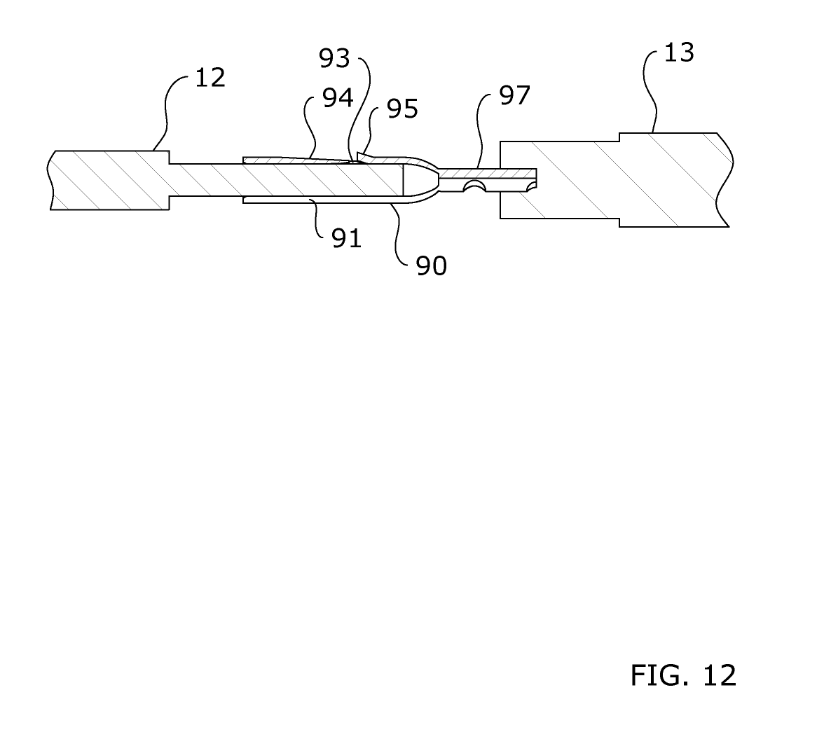

A preferred configuration of a contact 90 for use with the present invention is best shown in FIGS. 11-12. As shown, the contact 90 includes a receiver portion 91 and a lead portion 97 extending from the receiver portion 91. The receiver portion 91 is adapted to receive the leading end of the medical instrument 12 and thus includes a receiver opening 92 leading to a receiver channel 93 in which the medical instrument 12 is inserted.

The receiver portion 91 may include one or more resilient contacts 94 extending partially into the receiver channel 93 as best shown in FIG. 11. These resilient contacts 94 are comprised of resilient, conductive material such that, absent force, they extend partially into the receiver channel 93. When a medical instrument 12 is inserted therein as shown in FIG. 12, the resilient contacts 94 will bend or move outwardly to make contact with one or more fixed contacts 95 also positioned on the receiver portion 91. Thus, positioning a medical instrument 12 within the receiver channel 93 will force the resilient contacts 94 to close a conductive connection with the fixed contacts 95 and thus allow conductive connection between the receiver portion 91 and the medical instrument 12.

The lead portion 97 extends outwardly from the receiver portion 91 and is comprised of conductive material which allows conductive connection with the conduit 13 in the second channel 27. The conduit 13 may directly contact the lead portion 97 in some embodiments. In preferred embodiments shown in the figures, the lead portion 97 will interconnect with a connector hub 98.

The connector hub 98 will be positioned in the second channel 27 and be adapted to receive and conductively connect with the conduit 13 when inserted therein. The connector hub 98 may also be adapted to removably secure the conduit 13 within the second channel 27. In some embodiments, the conduit 13 may be fixedly secured therein, such as by having the connector hub 98 be fixedly secured within the housing 20 and integrally formed with the conduit 13.

### D. Medical Instrument/Conduit.

The present invention is adapted to interconnect a medical instrument 12 with a conduit 13 in a conductive manner. It should be appreciated that any type of medical instrument 12 or conduit 13 may be utilized with the present invention and the present invention should not be construed as limited to connecting any specific medical instrument 12 or conduit 13.

The conduit 13 may be connected to a power source or medical device. Thus, any arrangement of connector (multi-pin adapter, standard power plug, etc.) may be included on the distal end of the conduit 13 to connect the present invention to a wide range of power sources or medical devices. Thus, the present invention should not be construed as being limited to any particular type of conduit 13.

The medical instrument 12 may be comprised of any elongated medical instrument 12 which may be removably secured within the housing 20, such as probes used in surgery and the like. In a preferred embodiment, the present invention may be utilized in connection with a medical instrument 12 such as a catheter guide wire. However, the present invention should not be construed as being limited to any particular type of medical instrument 12.

### E. Operation of Preferred Embodiment.

In use, the conduit 13 will first be inserted through the second opening 26 of the housing 20 and seated within the connector hub 98 positioned at the distal end of the second channel 27. This step may be omitted in embodiments in which the conduit 13 is preinstalled within the housing 20.

The clamp 80 and arms 50, 60 must be opened before insertion of a medical instrument 12 within the housing 20. Thus, a user must first ensure the knob 70 is in its proper position to allow the clamp 80 and arms 50, 60 to be in their opened state and thus expose the receiver opening 87 of the clamp 80 and first opening 22 of the housing 20. If the clamp 80 and arms 50, 60 are in the closed position, the knob 70 is turned in a first direction to open the clamp 80 and arms 50, 60.

With the clamp 80 and arms 50, 60 opened, the medical instrument 12 may be inserted through the receiver opening 87 of the clamp 80 and inserted through the first channel 23 until it seats within the contact 90. The resilient contacts 94 of the contact 90 will be pressed outwardly to conductively connect with the fixed contacts 95 upon insertion of the medical instrument 12 within the receiver portion 91 of the contact 90. Thus, an electrical connection may be made between the medical instrument 12 and conduit 13 via the contact 90 and connector hub 98.

With the connection between the medical instrument 12 and conduit 13 established, the medical instrument 12 may be clamped into the housing 20 so that it is not inadvertently removed from conductive connection with the contact 90. The knob 70 is turned in a second direction, which will allow the inner lip 76 of the knob 70 to compress and close both the arms 50, 60 and the jaws 85, 86 of the clamp 80. The inner edges 57, 67 of the arms 50, 60 will close upon the medical instrument 12 within the first channel 23 to secure the medical instrument 12 therein. The jaws 85, 86 of the clamp 80 will also close down upon the medical instrument 12 to aid in retaining the medical instrument 12 within the housing 20. The conduit 13 may then be connected to a power source or medical device, and the present invention used for medical procedures (such as surgical procedures).

When completed, the knob 70 may be turned back in the first direction to release the clamp 80 and arms 50, 60. The medical instrument 12 may then be freely removed from the housing 20 and the present invention will be ready for future use with the same or a different medical instrument 12 using the preceding steps.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A medical instrument clamp, comprising:
a housing including a first end and a second end;
a contact internal to said housing;
a conduit extending into a first end of said housing, wherein said conduit is conductively linked with said contact;
a channel extending from a second end of said housing to said contact, wherein said contact is adapted to conductively link a medical instrument to said conduit; and
a clamp positioned at a first end of said housing for securing said medical instrument within said channel.

2. The medical instrument clamp of claim 1, wherein said housing includes a knob for adjusting said clamp between an opened state and a closed state.

3. The medical instrument clamp of claim 2, wherein said knob includes one or more raised splines extending along its outer circumference.

4. The medical instrument clamp of claim 2, wherein said knob includes a locking tab adapted to move within a stopper slot of said housing.

5. The medical instrument clamp of claim 4, wherein said locking tab and said stopper slot are operable to limit rotation of said knob.

6. The medical instrument clamp of claim 1, wherein said contact comprises a receiver portion and a lead portion extending from said receiver portion.

7. The medical instrument clamp of claim 6, wherein said receiver portion includes a receiver opening and a receiver channel adapted to receive said medical instrument.

8. The medical instrument clamp of claim 7, wherein said receiver portion includes at least one resilient contact and at least one fixed contact, wherein said at least one resilient contact is adapted to move into direct contact with said at least one fixed contact when said medical instrument is inserted into said receiver channel.

9. The medical instrument clamp of claim 8, wherein said conduit is conductively connected with said lead portion of said contact.

10. The medical instrument clamp of claim 8, wherein said lead portion of said contact is conductively connected to a connector hub.

11. The medical instrument clamp of claim 9, wherein said conduit is conductively connected to said connector hub.

12. The medical instrument clamp of claim 1, wherein said housing includes a first arm and a second arm positioned around said channel, wherein said first and second arms are operable to close around and secure said medical instrument within said channel.

13. The medical instrument clamp of claim 12, wherein said first arm includes a first anchor and wherein said second arm includes a second anchor.

14. The medical instrument clamp of claim 13, wherein said clamp includes a first receiver slot and a second receiver slot and wherein said first anchor is positioned within said first receiver slot and said second anchor is positioned within said second receiver slot.

15. The medical instrument clamp of claim 14, further comprising a knob positioned around said first arm and said second arm.

16. The medical instrument clamp of claim 15, wherein said knob includes an inner lip defining a lip opening, wherein said first arm and said second arm each extend through said lip opening, wherein said inner lip is operable to close said first arm against said second arm when said knob is in a first position.

17. The medical instrument clamp of claim 16, wherein said lip opening is comprised of an oval shape.

18. The medical instrument clamp of claim 16, wherein said lip opening is comprised of an octagonal shape.

19. The medical instrument clamp of claim 1, wherein said medical instrument is comprised of a catheter guide wire.

20. A medical instrument clamp, comprising:
a housing including a first end and a second end;
a contact internal to said housing, wherein said contact comprises a receiver portion and a lead portion extending from said receiver portion, wherein said receiver portion includes a receiver opening and a receiver channel adapted to receive a medical instrument, wherein said receiver portion includes at least one resilient contact and at least one fixed contact, wherein said at least one resilient contact is adapted to move into direct contact with said at least one fixed contact when said medical instrument is inserted into said receiver channel;
a conduit extending into a first end of said housing, wherein said conduit is conductively connected to a connector hub, wherein said lead portion of said contact is conductively connected to a connector hub;
a channel extending from a second end of said housing to said contact, wherein said housing includes a first arm and a second arm positioned around said channel, wherein said first and second arms are operable to close around and secure said medical instrument within said channel, wherein said first arm includes a first anchor and wherein said second arm includes a second anchor;
a knob positioned around said first arm and said second arm, wherein said knob includes an inner lip defining a lip opening, wherein said first arm and said second arm each extend through said lip opening, wherein said lip opening is comprised of an octagonal shape, wherein said knob includes one or more raised splines extending along its outer circumference, wherein said knob includes a locking tab adapted to move within a stopper slot of said housing, wherein said locking tab and said stopper slot are operable to limit rotation of said knob; and
a clamp positioned at a first end of said housing, wherein said clamp includes a first receiver slot and a second receiver slot, wherein said first anchor is positioned within said first receiver slot and said second anchor is positioned within said second receiver slot, wherein said clamp includes a first jaw and a second jaw for closing around said medical instrument.
